(19) United States
(12) Patent Application Publication
(10) Pub. No.: US 2017/0182085 A1
(43) Pub. Date: Jun. 29, 2017

(54) THERAPEUTIC AGENT FOR KERATOCONJUNCTIVE DISORDER

(71) Applicant: Santen Pharmaceutical Co., Ltd., Osaka-shi, Osaka (JP)

(72) Inventors: Shinichiro KANEKO, Kumamoto-shi, Kumamoto (JP); Masaaki SASAOKA, Ikoma-shi, Nara (JP); Takashi NAGANO, Ikoma-shi, Nara (JP); Satoshi SHIRAE, Ikoma-shi, Nara (JP)

(73) Assignee: Santen Pharmaceutical Co., Ltd., Osaka-shi, Osaka (JP)

(21) Appl. No.: 15/325,271

(22) PCT Filed: Jul. 13, 2015
(86) PCT No.: PCT/JP2015/069996

§ 371 Date: Jan. 10, 2017

(30) Foreign Application Priority Data
Jul. 14, 2014 (JP) 2014-144555

(51) Int. Cl. A61K 31/737; A61K 9/00

(52) U.S. Cl. CPC A61K 31/737; A61K 9/0048

(57) ABSTRACT

Provided are a therapeutic agent and a prophylactic agent for keratoconjunctive disorder, each agent comprising sacran. A therapeutic agent or a prophylactic agent for keratoconjunctive disorder, each agent comprising sacran.

### TECHNICAL FIELD

The present invention relates to a therapeutic agent and a prophylactic agent for keratoconjunctive disorders, each agent comprising sacran.

### BACKGROUND ART

Sacran is known as a polysaccharide derived from Aphanothece sacrum. As applications of sacran, polarization lenses as a polysaccharide-metal ion hybrid liquid crystal gel, photo-trapping agents, photo-scattering agents, flow rate sensors, play toys, actuators, and adhesives have been anticipated, and an anti-oxidation action, an anti-allergic activity and an anti-bacterial property have been reported regarding Aphanothece sacrum, thus, applications such as anti-viral agents, anti-cancer agents, health promotion agents for use in foods, thickeners and stabilizers for foods, and the like have been anticipated (for example, PTL 1).

A gel-like sustained release carrier and a drug delivery system utilizing the carrier to release the drug has been proposed as an application of sacran (for example, PTL 2).

It has been suggested that sacran has numerous possible applications, and thereamong, it is desirable to search for novel pharmacological applications.

Patent Document 1: PCT International Publication No. WO2008/062574

Patent Document 2: Japanese Unexamined Patent Application, Publication No. 2011-68606

### DISCLOSURE OF THE INVENTION

### Problems to be Solved by the Invention

It is the object of the present invention, taking the aforementioned circumstances into consideration, to provide a therapeutic agent and a prophylactic agent for a keratoconjunctive disorder, each agent comprising sacran.

### Means for Solving the Problems

The present inventors discovered that sacran brings about an excellent improvement effect for corneal disorders in a therapeutic effects test of dry eye and other keratoconjunctive disorders, and completed the present invention.

Specifically, the present invention provides the following.

[1] A therapeutic agent or a prophylactic agent for keratoconjunctive disorder, each agent comprising sacran.

[2] The therapeutic agent or the prophylactic agent of [1], wherein the keratoconjunctive disorder is at least one selected from the group consisting of keratitis, conjunctivitis and keratoconjunctivitis.

[3] The therapeutic agent or the prophylactic agent of [1], wherein the keratoconjunctive disorder is at least one selected from the group consisting of dry eye, corneal ulcer, corneal erosion, superficial punctate keratopathy, corneal epithelial defect, conjunctival epithelial defect, conjunctive epithelium disorder, keratoconjunctivitis sicca, superior limbic keratoconjunctivitis and filamentary keratitis.

[4] The therapeutic agent or the prophylactic agent according to any of [1] to [3], which is in the dosage form of an ophthalmic solution or an ophthalmic ointment.

### Effects of the Invention

According to the present invention, by comprising sacran, a therapeutic agent and a prophylactic agent for a keratoconjunctive disorder having excellent water storing and water supplying properties when administered to the eye can be provided. Specifically, a therapeutic effects test of corneal disorders was performed, and the sacran in the present invention brings about an excellent therapeutic promoting effect in a corneal disorder model, and thus, effectively operates as a therapeutic agent and a prophylactic agent for keratitis such as corneal ulcer, corneal erosion, superficial punctate keratopathy, and corneal epithelial defect; conjunctivitis such as conjunctival epithelial defect and conjunctive epithelium disorder; and keratoconjunctivitis such as keratoconjunctivitis sicca (dry eye), superior limbic keratoconjunctivitis, and filamentary keratitis.

### BRIEF DESCRIPTION OF THE DRAWINGS

FIG. 1 is a drawing relating to the determination criteria of the therapeutic effects test of corneal disorders and shows the division of the cornea and the score.

### PREFERRED MODE FOR CARRYING OUT THE INVENTION

An embodiment of the present invention will be explained below.

The therapeutic agent and the prophylactic agent for a keratoconjunctive disorder of the present invention comprise sacran. Sacran is one type of sulfated polysaccharide, has a molecular weight of 2,000,000 or more, and the CAS Registry Number is 1039552-36-7. Sacran, specifically, has a repeat structure of a sugar chain unit where a sugar constituent having a hexose structure and a sugar constituent having a pentose structure are conjugated together in a linear chain or a branched chain through an α-glycoside bond or a β-glycoside bond, the sugar chain unit contains a lactated, sulfated sugar as the sugar constituent, and, in the sugar chain unit, 2.7 or more hydroxyl groups per 100 hydroxyl groups are sulfated or sulfur elements occupy 1.5% by weight or more of all of the elements.

It is known that sacran is obtained by protonating a polysaccharide extracted from Aphanothece sacrum which is an endemic species in Japan, and performing partial hydrolysis thereon (for example PTL 1).

Sacran which can be used in the present invention is not limited to sacran derived from Aphanothece sacrum.

In the present invention, the keratoconjunctive disorders indicate a state in which the cornea and the conjunctiva have received damage due to various factors. Keratoconjunctive disorders are caused by a reduction in the lacrimal gland due to, for example, dry eye (keratoconjunctivitis sicca), and are caused by injury, autoimmune diseases, drug toxicity and the like.

Keratoconjunctive disorders, from the viewpoint of symptoms, are roughly categorized into keratitis such as corneal ulcer, corneal erosion, superficial punctate keratopathy, and corneal epithelial defect; conjunctivitis such as conjunctival epithelial defect, and conjunctive epithelium disorder. Keratoconjunctive disorders, from the viewpoint of the cause, the site on onset and the symptoms, include keratoconjunctivitis such as keratoconjunctivitis sicca, superior limbic keratoconjunctivitis, and filamentary keratitis. The therapeutic agent and the prophylactic agent for the keratoconjunctive disorders of the present invention can be used in all kinds of keratoconjunctive disorders, and thereamong, keratoconjunctivitis sicca is preferable. Keratoconjunctivitis sicca is also known as dry eye.

The therapeutic agent and the prophylactic agent for a keratoconjunctive disorder of the present invention can be administered parenterally. An ophthalmic solution, an ophthalmic ointment, injections, and the like may be provided as the dosage form, and thereamong, an ophthalmic solution and an ophthalmic ointment are preferred, and the ophthalmic solution is more preferred.

These dosage forms can be prepared using any of the generally used techniques.

For example, the ophthalmic solution can be prepared by using a tonicity agent such as sodium chloride and concentrated glycerin; a buffer agent such as sodium phosphate and sodium acetate; a surfactant such as polyoxyethylene sorbitan monoolate, polyoxyl 40 stearate and polyoxyethylene hydrogenated castor oil; a stabilizing agent such as sodium citrate and sodium edetate; a preservative such as benzalkonium chloride and paraben; and the like as necessary. The pH may be within a range acceptable for ophthalmic drug products, and preferably is in the range of 4 to 8.

The ophthalmic ointment can be prepared with a generally used base such as white petrolatum or liquid paraffin.

The therapeutic agent and the prophylactic agent for a keratoconjunctive disorder of the present invention is useful as a prophylactic agent, but preferably is a therapeutic agent.

In the therapeutic agent and the prophylactic agent for a keratoconjunctive disorder of the present invention, specifically, the dosage of the therapeutic agent can be appropriately selected depending on the symptoms, age, the dosage form, and the like, but in an ophthalmic solution, the sacran concentration may be instilled at 0.00001 to 1% (w/v), preferably 0.0001 to 1% (w/v) one to several times per day, and specifically, one to six times per day, and two or three times per day is preferable.

### EXAMPLES

The sacran preparation example, the formulation examples and the results of the pharmacological test are shown below, but these examples are provided for the purpose of better understanding of the present invention, and do not limit the scope of the present invention.

### Preparation Example of Sacran

The water-soluble pigment was removed by freezing an appropriate amount Aphanothece sacrum and water washing after melting, and next, the liposoluble pigment was removed while stirring overnight in ethanol. After separating the ethanol from Aphanothece sacrum from which these pigments were removed, the solution was heated in a 0.4-0.6N aqueous solution of sodium hydroxide at 40° C., and dissolved while stirring over approximately five hours. An aqueous solution of the sugar derivative extracted by the operation was neutralized with hydrochloric acid and desalinated by soaking in 60 to 80% isopropyl alcohol. After desalinization, the concentrate was poured into 100% isopropanol under agitation and a gel-like sacran precipitated, and the gel-like sacran was dried by hot air at 85° C. or higher for six hours to obtain sacran (the present compound) derived from Aphanothece sacrum which is a fibrous sugar derivative.

### Formulation Examples

Typical formulation examples which used sacran (the present compound) are shown below.

### [Formulation Example 1] Ophthalmic Solution

In 100 ml

| | |
|---|---|
| Present compound | 200 mg |
| Sodium chloride | 900 mg |
| Sterile purified water | Appropriate amount |

### [Formulation Example 2] Ophthalmic Solution

In 100 ml

| | |
|---|---|
| Present compound | 200 mg |
| Sodium chloride | 800 mg |
| Disodium hydrogenphosphate | 100 mg |
| Sodium dihydrogenphosphate | Appropriate amount |
| Sterile purified water | Appropriate amount |

### [Formulation Example 3] Ophthalmic Ointment

In 100 g

| | |
|---|---|
| Present compound | 0.2 g |
| Liquid paraffin | 10.0 g |
| White petrolatum | Appropriate amount |

### [Pharmacological Test]

### 1. Therapeutic Effects Test of Corneal Disorders

Using a male SD rat, a corneal disorder model was produced according to the method of Fujihara, et al. (Invest. Ophthalmol. Vis. Sci 42(1): 96-100(2001)). After producing the corneal disorder model, the extent of the corneal disorder was scored according to the method of Murakami, et al. (Atarashii Ganka (New Ophthalmology) 21(1):87-90. (2004)).

### (Test Method)

A male SD rat was systematically anesthetized by an interperitoneal administration of 2.5 mL/kg Somnopentyl injection diluted five-fold with a physiological saline solution, and maintained in the anesthetized state by inhalation of (1.5%) Escain. Next, an incision was made on both sides of the buccal skin, the exorbital lacrimal gland was removed, and the skin was sutured by a Michel suture clip. Then, corneal damage was induced over eight weeks. Further, normal animals in which the removal of the lacrimal gland was not performed were prepared.

Next, 5 μL of the phosphate buffer solution (PBS) was instilled six times per day for four weeks in both eyes (normal eye) in the normal animals, and 5 μL of PBS (control), or a 0.006% or a 0.2% PBS solution of the present compound were instilled six times per day for four weeks in both eyes in the corneal disorder model.

The cornea was stained with fluorescein prior to and four weeks after the initiation of instillation. The upper, middle and lower parts of the cornea were assessed for the extent of staining with fluorescein according to the following criteria, and the extent of the corneal damage was calculated from the mean value of the total scores of the aforementioned respective parts.

The normal eyes were tested in the same manner as stated above and the mean value of the total scores of the aforementioned respective parts was sought.

### (Determination Criteria)

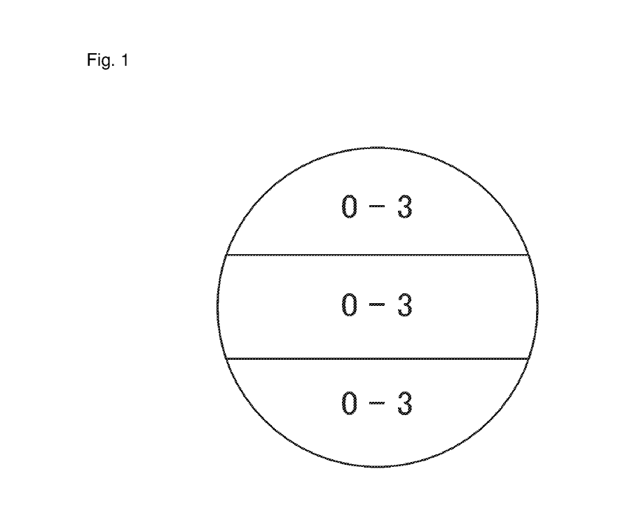

1) The cornea was divided into upper, middle and lower parts as shown in FIG. 1, and a score from 0 to 3 was conferred on the basis of the criteria of Table 1. 0.5 was provided as an intermediate value.
2) The scores of the three regions were totaled, and a defect score (0-9) was calculated for each observed eye.

TABLE 1

| Score | Determination criteria |
|---|---|
| 0 | No punctate staining |
| 1 | Scattered staining |
| | (Fluorescein staining of punctates |
| | separated) |
| 2 | Intermediate staining |
| | (Intermediate between 1 and 3) |
| 3 | Heavy staining |
| | (Fluorescein staining of punctates |
| | nearly abut) |

### (Results)

The improvement ratio due to the instillation of the present compound calculated on the basis of the mean value of the total scores of the aforementioned respective parts in the control group as a standard value (improvement ratio: 0%) according to the following calculation formula is shown in FIG. 2. Note that, the mean value of the scores is the average of 8 eyes (4 animals).

Improvement ratio (%)={(control)−(present compound)}/damage degree×100

Damage degree={(control)−(normal eye)}

TABLE 2

Four weeks after start of
ocular instillation

| | Mean value of | Improvement |
|---|---|---|
| Group | total scores | ratio (%) |
| Normal eye | 2.7 | |
| Control | 5.6 | 0 |
| 0.006% of present | 4.4 | 41 |
| compound | | |
| 0.2% of present | 2.7 | 100 |
| compound | | |

### (Discussion)

As is apparent from the results of the pharmacological test in which the aforementioned rat was used, the present compound markedly improves the corneal disorders.

### 2. Therapeutic Effects Test of Corneal Disorders (Examination of the Instillation Frequency)

The aforementioned test results were examined by the same test method regarding the influence of the instillation frequency of a 0.2% PBS solution of the present compound on the improvement result on corneal disorders.

### (Test Method)

Corneal damage was induced over eight weeks by performing the same operation as in the aforementioned test to the male SD rats. Further, normal animals in which the removal of the lacrimal glands was not performed were prepared.

Next, 5 μL of PBS (normal eye) was instilled six times per day for four weeks in both eyes in the normal animal, 5 μL of PBS (control) was instilled six times per day for four weeks in both eyes and 5 μL of 0.2% PBS solution of the present compound was instilled two or six times per day for four weeks in both eyes in the corneal disorder model.

The cornea was stained with fluorescein prior to and one, two, and four weeks after the initiation of instillation, and the extent of the corneal disorder was calculated by the same methods as the aforementioned test.

The normal eye was tested in the same manner as stated above and the mean value of the total scores of the aforementioned respective parts was sought.

### (Results)

The improvement ratio due to the instillation of the present compound calculated in the same manner as the improvement effect in the aforementioned test is shown in Table 3. Note that, the mean value of the scores is the average of 8 eyes (4 animals).

TABLE 3

| | One week after | | Two weeks after | | Four weeks after | |
|---|---|---|---|---|---|---|
| | start of ocular | | start of ocular | | start of ocular | |
| | instillation | | instillation | | instillation | |
| | Mean | Im- | Mean | Im- | Mean | Im- |
| | value | prove- | value | prove- | value | prove- |
| | of | ment | of | ment | of | ment |
| | total | ratio | total | ratio | total | ratio |
| Group | scores | (%) | scores | (%) | scores | (%) |
| Normal eye | 2.4 | | 3.5 | | 3.6 | |
| Control | 5.8 | 0 | 5.2 | 0 | 6.0 | 0 |
| Ocular | 4.0 | 53 | 3.5 | 100 | 3.8 | 92 |
| instillation | | | | | | |
| two times | | | | | | |
| per day | | | | | | |
| Ocular | 3.4 | 71 | 3.3 | 112 | 3.0 | 125 |
| instillation | | | | | | |
| six times | | | | | | |
| per day | | | | | | |

### (Discussion)

As is apparent from the results of the pharmacological test in which the aforementioned rat was used, the present compound markedly improves the corneal disorders, and the improvement effect was observed even with ocular instillation two times per day, but specifically, the improvement effect could be observed from the early stage when ocular instillation was performed six times per day.

### 3. Evaluation Testing of the Conjunctive Epithelium Disorder Improvement Action

The aforementioned test results were examined regarding the improvement effect on conjunctive epithelium disorder (reduction of conjunctival goblet cells) of a 0.2% PBS solution of the present compound.

### (Test Method)

Corneal damage was induced over eight weeks by performing the same operation as in the aforementioned test to the male SD rats. Further, normal animals in which the removal of the lacrimal glands was not performed were prepared.

Next, 5 μL of PBS (normal eye) was instilled six times per day for four weeks in both eyes in the normal animal, 5 μL of PBS (control) was instilled six times per day for four weeks in both eyes and 5 μL of 0.2% PBS solution of the present compound was instilled two or six times per day for four weeks in both eyes in the conjunctive epithelium disorder model.

The eyelids were excised four weeks after the start of ocular instillation and a histopathologic examination was performed. After the excised eyelids were immersed and fixed in 10% neutral buffered formalin, and the paraffin substituted, the ear sides were made as an embedded surface to prepare a block, and thin cutting was performed to produce a PAS reaction sample.

Using a 40× objective lens, the number of all of the conjunctival goblet cells observed in the samples were counted for the respective upper and lower eyelids.

### (Results)

The effect for increasing the conjunctival goblet cells by continuous instillation of the present compound for four weeks are shown in FIG. 4. Note that, the mean value of the number of goblet cells is the average of 7-8 eyes (4 animals).

TABLE 4

| | | Number of goblet cells |
|---|---|---|
| Group | Number | (Mean value) |
| Normal eye | 8 eyes | 583 |
| Control | 8 eyes | 446 |
| 0.2% of present | 7 eyes | 551 |
| compound | | |
| Ocular instillation | | |
| two times per day | | |
| 0.2% of present | 7 eyes | 514 |
| compound | | |
| Ocular instillation | | |
| six times per day | | |

### (Discussion)

From the aforementioned histopathological test results, the present compound has an effect for improving conjunctive epithelium disorders caused by the removal of the lacrimal glands, and this improvement effect was observed not only with ocular instillation six times per day, but also with ocular instillation two times per day.

1. A method of treating or preventing keratoconjunctive disorder comprising: administering to a subject effective amount of sacran.

2. The method according to claim 1, wherein the keratoconjunctive disorder is at least one selected from the group consisting of keratitis, conjunctivitis and keratoconjunctivitis.

3. The method according to claim 1, wherein the keratoconjunctive disorder is at least one selected from the group consisting of dry eye, corneal ulcer, corneal erosion, superficial punctate keratopathy, corneal epithelial defect, conjunctival epithelial defect, conjunctive epithelium disorder, keratoconjunctivitis sicca, superior limbic keratoconjunctivitis and filamentary keratitis.

4. The method according to claim 1, wherein sacran is in the dosage form of an ophthalmic solution or an ophthalmic ointment.

5. The method according to claim 2, wherein sacran is in the dosage form of an ophthalmic solution or an ophthalmic ointment.

6. The method according to claim 3, wherein sacran is in the dosage form of [symbol]
